(19) United States
(12) Patent Application Publication
(10) Pub. No.: US 2018/0318563 A1
(43) Pub. Date: Nov. 8, 2018

(54) COLLAR AND BRACKET FOR AN INFLATION DEVICE

(71) Applicant: Cook Medical Technologies LLC, Bloomington, IN (US)

(72) Inventors: Hilbert D. Brown, Winston-Salem, NC (US); Scott D. Reed, Advance, NC (US)

(73) Assignee: Cook Medical Technologies LLC, Bloomington, IN (US)

(21) Appl. No.: 15/969,588

(22) Filed: May 2, 2018

Related U.S. Application Data
(60) Provisional application No. 62/502,211, filed on May 5, 2017.

(51) Int. Cl. A61M 25/10; A61M 29/02

(52) U.S. Cl. CPC A61M 25/10182; A61M 2210/1042; A61M 2205/3331; A61M 29/02

(57) ABSTRACT

In one aspect, the present disclosure provides a collar for securement to a barrel of a syringe, the barrel having an outer diameter and a flange extending from the outer diameter. The collar may include a distal opening with an inner diameter configured to surround the outer diameter of the barrel, a collar flange located proximal the distal opening and having a length and a thickness greater than a length and a thickness of the flange of the barrel, and a flange-receiving cavity extending at least partially within the collar flange and configured to receive the flange of the barrel.

### RELATED APPLICATION

This application claims the benefit of U.S. Provisional Application No. 62/502,211, filed May 5, 2017, which is hereby incorporated by reference in its entirety.

### BACKGROUND

Balloon dilation is a medical procedure that generally includes the use of a catheter with an inflatable balloon at its distal tip. The inflatable balloon may be maneuvered into a body opening or cavity while deflated. Once positioned, the balloon may be inflated to dilate the body opening or cavity.

This procedure may be used in a variety of medical applications. For example, an inflatable balloon of an inflation catheter (also referred to as a “balloon dilator”) may be deployed within the vascular system to repair abnormal narrowing or blockages of blood vessels (i.e., stenosis) caused by buildup of fatty deposits, calcium build-up (atherosclerosis), etc. In another application, an inflation catheter may be used in the ureter to facilitate the removal of kidney stones. A similar device may be used in the gastrointestinal tract to repair achalasia, for example. In one particular procedure, an inflatable balloon can be deployed and then inflated to tear (in a controlled manner) the spastic muscle at the end of the esophagus.

The inflation catheter may be attached to an inflation system, which may be located at the proximal end of the catheter at a location outside the body of the patient and accessible to a medical professional. The inflation system may include a syringe with a standard-sized barrel and a standard-sized plunger. While the medical professional may operate the syringe by manually moving the plunger within the barrel, the medical professional may instead control the plunger position with a separate device, such as a ratchet device or other type of actuation device. The ratchet device or other actuation device may allow for precise one-handed control over the plunger position within the barrel (and therefore precise control over the inflation of the balloon). Existing actuation devices are typically designed for a syringe of a particular size. Thus, it would be advantageous to provide a device allowing the actuation device to be used with different sized syringes.

### DESCRIPTION

In one aspect, the present disclosure provides a collar for securement to a barrel of a syringe, the barrel having an outer diameter and a flange extending from the outer diameter. The collar may include a distal opening with an inner diameter configured to surround the outer diameter of the barrel, a collar flange located proximal the distal opening and having a length and a thickness greater than a length and a thickness of the flange of the barrel, and a flange-receiving cavity extending at least partially within the collar flange and configured to receive the flange of the barrel.

The collar may include an outer diameter surface with a larger diameter than the outer diameter of the barrel of the syringe.

The collar flange may be a first collar flange, where a first half includes the first collar flange and a second half includes a second collar flange. The first half and the second half may be discrete elements configured to secure together around the outer diameter of the syringe.

The collar may include an outer diameter surface with a diameter of about 29.5 mm. A length of the collar flange may be about 10.4 mm. A thickness of the collar flange may be about 3.8 mm.

In another aspect, the present disclosure provides an in inflation system for an inflation catheter. The inflation system may include a syringe, the syringe having a barrel with a cylindrical chamber and a plunger. The plunger may have a distal end being slidable within the cylindrical chamber of the barrel, where a flange of the syringe extends from an outer diameter of the barrel. The inflation system may further include a collar, the collar having a flange-receiving cavity for receiving the flange of the syringe and an opening with an inner diameter surrounding the outer diameter of the barrel.

The collar may include an outer diameter surface with a larger diameter than an outer diameter of the barrel of the syringe.

The collar may include a first half with a first collar flange and a second half with a second collar flange, where the first half and the second half are discrete elements configured to secure together.

A collar flange of the collar may be configured to be received by an opening of a bracket of an actuation device. The inflation system may further include the bracket, where the bracket includes a detent extending into the opening of the bracket.

A spacer may be included and configured to be positioned between the barrel of the syringe and a top surface of the actuation device.

The collar may include an outer diameter surface with a diameter of about 29.5 mm. A length of the collar flange may be about 10.4 mm. A thickness of the collar flange may be about 3.8 mm.

In another aspect, the present disclosure provides another embodiment of an inflation system of an inflation catheter. The inflation system may include a syringe, the syringe having a barrel with a cylindrical chamber and a plunger, the plunger having a distal end being slidable within the cylindrical chamber of the barrel, and a flange of the syringe extending from an outer diameter of the barrel. A collar may be included with a collar flange, the collar having an opening configured to receive an outer diameter of the barrel of the syringe. An actuation device may be included and configured to move the plunger with respect to the barrel of the syringe, the actuation device including a bracket with a proximal element, a distal element, and an opening between the proximal element and the distal element. The collar flange may be received by the opening of the bracket.

The distal element of the bracket may include a detent extending into the opening of the bracket.

The collar may include a flange-receiving cavity for receiving a flange of the syringe.

A spacer may be located between a top surface of the actuation device and the barrel of the syringe.

The collar flange may be a first collar flange, the collar further including a first half with the first collar flange and a second half with the second collar flange, where the first half and the second half are discrete elements configured to secure together.

### BRIEF DESCRIPTION OF THE DRAWINGS

FIG. 1 shows a side view of an embodiment of an inflation system for an inflation catheter in accordance with the present disclosure.

FIG. 2 shows a top perspective view of the inflation system of FIG. 1.

FIG. 3 shows the syringe of FIG. 1 with a collar providing a flange for compatibility with an actuation device in accordance with the present disclosure.

FIG. 4 shows a front, distal-facing view of the collar of FIG. 3.

FIG. 5 shows a side view of the collar of FIG. 3 and FIG. 4.

FIG. 6 shows the collar of FIGS. 3-5 when manufactured as two discrete pieces in accordance with the present disclosure.

FIG. 7 shows another embodiment of an inflation system, where an actuation device includes a bracket having a detent in accordance with the present disclosure.

### DETAILED DESCRIPTION

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the medical device to a patient. Hence the term “distal” means the portion of the medical device that is farthest from the physician and the term “proximal” means the portion of the medical device that is nearest to the physician.

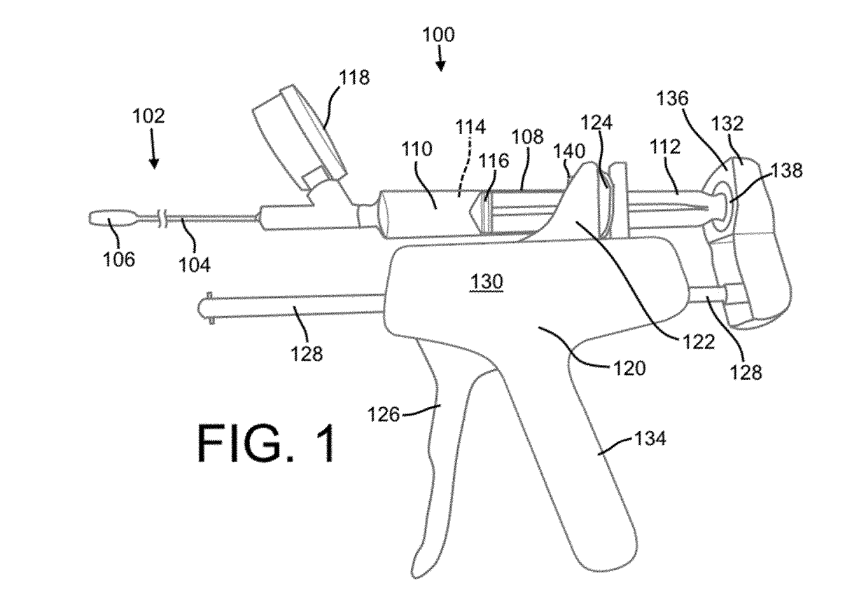

FIG. 1 shows a side view of an inflation system 100 coupled to an inflation catheter 102. Referring to FIG. 1, the inflation catheter 102 may include a tube portion 104 with an inflatable portion at its distal end. The inflatable portion, which may also be referred to as a balloon 106, may expand radially when pressurized. The tube portion 104 may provide fluid communication between the balloon 106 and the inflation system 100. As described in more detail below, the inflation system 100 may be configured to force a fluid (e.g., water or saline) through the tube portion 104 and into the balloon 106 such that the balloon 106 dilates a body opening or cavity.

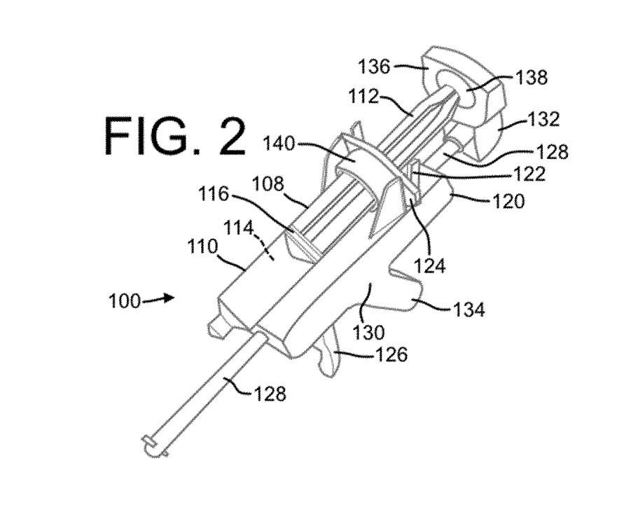

FIG. 2 shows a top perspective view of the inflation system of FIG. 1. As shown in FIGS. 1-2, the inflation system 100 may include a syringe 108 having a syringe barrel 110 and a plunger 112. The barrel 110 may include a cylindrical chamber 114 in fluid communication with the inflation catheter 102 and configured (e.g., sized and shaped) to receive the plunger 112. The plunger 112 may have a distal end 116 formed of a compliant material (e.g., a rubber) such that the plunger provides a seal against the inner diameter of the barrel 110. When the plunger 112 moves distally, the plunger 112 may increase the pressure of a fluid within the chamber 114, which may operate to force the liquid (or other fluid) out of the chamber 114 and into the inflation catheter 102. It may be advantageous to use a substantially incompressible liquid (like water or saline) such that the balloon responds almost immediately to movement of the plunger.

Inflation balloons are used for a variety of medical procedures, and different applications call for different balloon sizes. For example, when used for applications in the esophagus, a balloon that is approximately 8 cm long and expandable to a maximum diameter of about 8 mm to about 20 mm may be used. In the pylorus, a balloon that is approximately 5.5 cm long and expandable to a maximum diameter of about 8 mm to about 20 mm may be used. Balloons in this size range may be compatible with a syringe that has dimensions typical of a commercially-available 60 cc syringe. In other medical applications, a smaller balloon may be used. For example, when used to treat certain medical conditions in the bowel duct, a balloon may be used that is about 3 cm in length and expandable to a maximum diameter of about 4 mm to about 10 mm depending on the particular application.

A specified pressure is associated with each size of balloon, and typically the smaller the balloon, the higher the specified pressure. Because the 60 cc syringe has a relatively large barrel diameter, a relatively high force on the plunger may be necessary to achieve a high specified pressure. Thus, it is advantageous (and potentially necessary) to use a syringe with a smaller barrel diameter when working with smaller balloons with relatively high specified pressures. For example, balloons such as those described above for use in the bowel duct may work particularly well with a syringe having dimensions similar to a commercially-available 20 cc syringe (which has a smaller diameter than the 60 cc syringe). Further, since the smaller syringe will be associated with a smaller liquid displacement per unit of plunger displacement, using a 20 cc syringe may increase precision in inflating a small balloon relative to the larger 60 cc syringe. While this disclosure refers repeatedly to 60 cc and 20 cc syringes, is not limited to syringes of those sizes, and the characteristics described herein (both with respect to the syringes themselves and the associated devices) are also applicable with respect to larger and/or smaller syringes (e.g., a 10 cc syringe or a 5 cc syringe).

In some embodiments, a pressure gauge 118 (shown in FIG. 1 but not FIG. 2) or other sensor may be in fluid communication with at least one of the syringe 108 and the inflation catheter 102. As depicted, for example, the pressure gauge 118 may be located between the syringe 108 and the inflation catheter 102. The pressure gauge 118 may determine a precise pressure of the fluid within the chamber 114 and/or inflation catheter 102 and relay that information to a medical professional. Since the pressure of the fluid may be directly correlated with the size of the balloon 106, the pressure gauge 118 may be advantageous for providing the medical professional with a real-time indication of the size of the balloon 106 and/or the percentage of inflation of the balloon 106 without necessitating a live image or video feed within the body by way of a scope, though it is contemplated that a scope could also be included with (or separate of) the inflation catheter 102.

The inflation system 100 may include an actuation device 120 configured to allow a medical professional to move the plunger 112 with respect to the barrel 110 with relatively-high precision. The actuation device 120 may have a bracket 122 configured (e.g., sized and shaped) to receive a flange 124 coupled to the syringe barrel 110. When the actuation device is a ratchet device, a one-hand operable trigger 126 may effect movement a shaft 128 (which extends through a body 130). When the actuation device 120 is in a first setting, pulling the trigger 126 may cause the shaft 128, and therefore the backstop 132, to move distally. The backstop 132 may push distally on the plunger 112 of the syringe 108 to increase pressure within the barrel 110. Thus, pulling the trigger 126 may cause the balloon 106 to inflate. In a second setting, pulling the trigger 126 may cause the shaft 128 to move backwards (e.g., proximally). It is contemplated that more than two settings may be included, and while not shown, a switch for selecting a particular setting may be included on the body. For example, in a third setting, the shaft 128 may disengage with the trigger 126 such that shaft is freely slidable along its longitudinal axis with respect to the body 130 without trigger movement.

The backstop 132 may have an engagement portion 136 shaped to receive a proximal end 138 (typically referred to as the thumb rest) of the plunger 112. In some embodiments, the engagement portion 136 may have a circular cavity with a diameter sized about the same as, or slightly larger than, a diameter of the proximal end 138 of the plunger 112. It is contemplated that the engagement portion 136 may be securable to the proximal end 138 of the plunger 112 such that it is capable of pulling the plunger 112 proximally when the actuation device 120 is in the above-described second setting.

The actuation device 120 may be a relatively high-cost component when compared to the syringe 108. Also, certain actuation devices may be designed and marketed for a specific medical procedure that is associated with a syringe of a certain size. For example, one particular actuation device may be designed to hold a 60 cc syringe, which may be suitable for compatibility with an inflation catheter typically used to dilate strictures in the upper gastrointestinal tract, colon, and/or pylorus. Because smaller actuation devices may not be available, and because of their relatively high cost, it would be advantageous to use that same actuation device with a smaller syringe (e.g., a 20 cc syringe) since the smaller syringe. However, existing actuation devices may have only one bracket 122 sized to receive a standard-sized flange of a particular commercially available syringe, such as a flange of a standard 60 cc syringe. Since smaller syringes typically have smaller flanges, a smaller syringe may not fit properly within the bracket 122 of the actuation device 120. To solve this problem, the flange 124 depicted in FIG. 1 may be a flange of a collar 140 that fits around an outer diameter of the barrel 110.

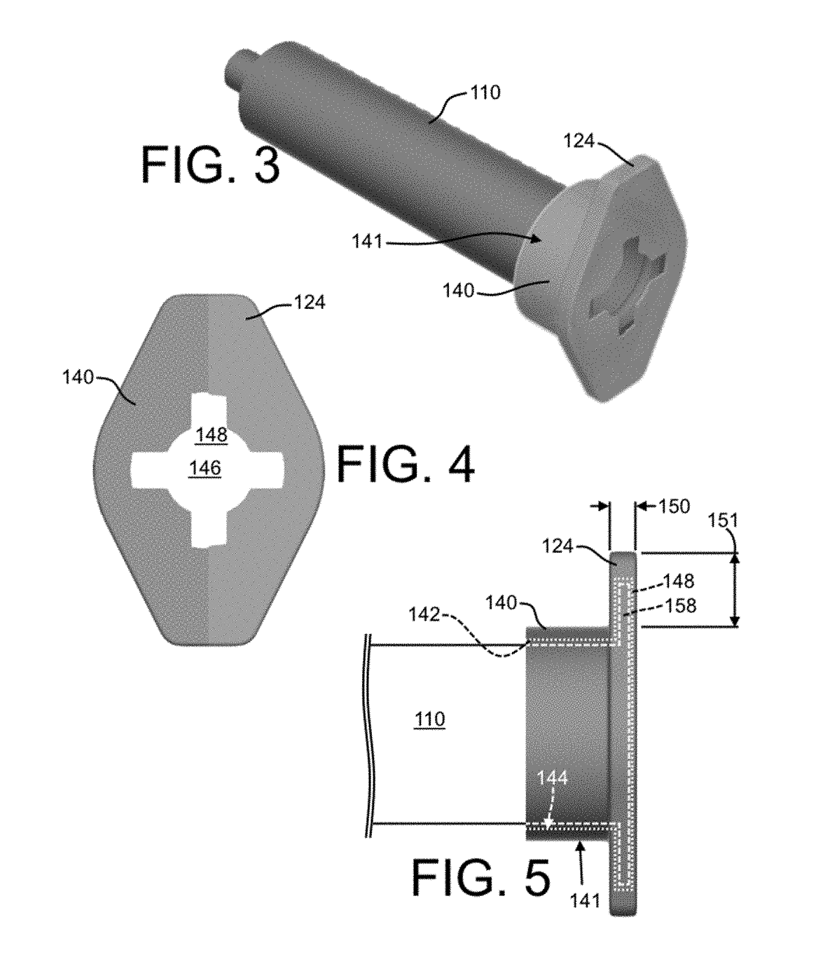

FIG. 3 shows the collar 140 with the flange 124, where the collar 140 is secured around the outer diameter of the barrel 110. FIG. 4 shows a front (i.e., distal-facing) view of the collar 140, and FIG. 5 shows a side view of the collar 140. Referring to FIGS. 3-5, the flange 124 may be approximately sized to mimic a flange of a particular syringe (such as a 60 cc syringe). For example, when the collar 140 is configured to mimic a 60 cc syringe (and thus fit properly within a bracket sized for a 60 cc syringe), the collar 140 may have an outer diameter surface 141 with a diameter of about 29.5 mm (1.16 in), the flange 124 may have a thickness 150 of about 3.8 mm (0.15 in), and the flange 124 may have a length 151 of about 10.4 mm (0.41 in) (measured from the nearest point of the outer diameter to the tip of the flange). Other suitable dimensions are also contemplated. Advantageously, the collar 140 may be relatively low-cost when compared to an actuation device, and may allow an actuation device designed for larger syringes to operate with a smaller syringe without the need for a syringe with a custom flange and/or barrel diameter.

The collar 140 may have a distal opening 142 with an inner diameter 144 that is about the same size as (or slightly larger than) the outer diameter of the barrel 110 (of FIG. 3). For example, when the collar 140 is configured to operate with a 20 cc syringe, the inner diameter 144 may be about 22.4 mm (0.88 in). In some embodiments and as shown in FIG. 4, the collar 140 may have a proximal opening 146 approximately matching a cross-sectional shape of a plunger, such as a cross pattern (which is typical of commercially available plungers). Other proximal opening shapes are also contemplated.

Referring to FIG. 5, the collar may include a flange-receiving cavity 148 for receiving the flange 158 of the barrel 110 (referred to herein as the original flange of the barrel). The flange-receiving cavity 148 may be sized with the approximate dimensions of the original flange, and thus may have a thickness 150 of about, or slightly larger than, 3.8 mm (0.15 in) when the syringe is a 20 cc syringe. While the flange-receiving cavities 148 may be advantageous for substantially fixing the collar 140 with respect to the barrel 110 (e.g., so the collar 140 cannot slide along the barrel 110), it is also contemplated that the collar 140 may not cover the flange 158 of the barrel 110 in certain embodiments.

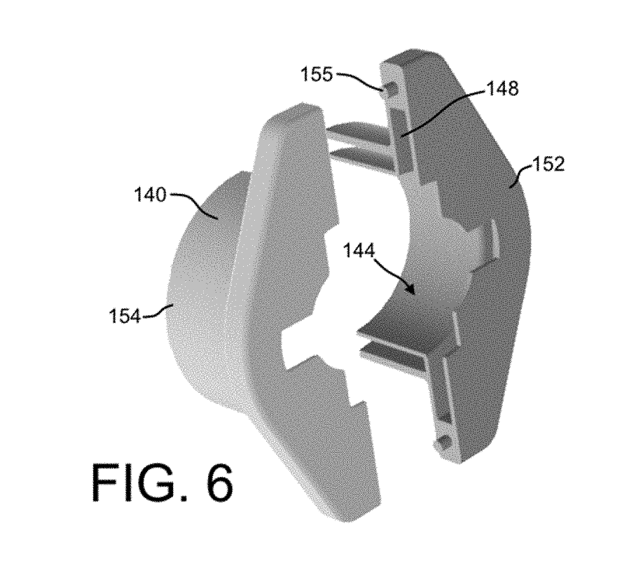

Referring to FIG. 6, the collar 140 may be manufactured as two (or more) discrete pieces/elements, and any suitable manufacturing process may be used. For example, a first half 152 and a second half 154 of the collar 140 may be separately molded (or otherwise formed separately). The first half 152 and the second half 154 may be configured to be secured together. For example, the first half 152 may include an extension 155 that is received by a cavity of the second half 154. An adhesive, a fastening device (e.g. a clamp, screw, or the like), and/or any other suitable securement device or method may be used to facilitate securement of the first half 152 and the second half 154. Advantageously, the first half 152 and the second half 154 may be assembled around the proximal end of the syringe barrel (see FIG. 3 and FIG. 5). This may allow the collar 140 to fit around the outer diameter of the barrel 110 without necessitating sliding the collar 140 onto the barrel (which may be difficult due to friction, particularly if an outer diameter of the barrel 110 is about the same as the inner diameter 144 of the collar 140). This may also provide the ability for the collar 140 to be placed over the original flange of the barrel 110 when the collar 140 has the above-described flange-receiving cavities 148. In some embodiments, the collar 140 may be removable from the barrel 110 such that it is reusable.

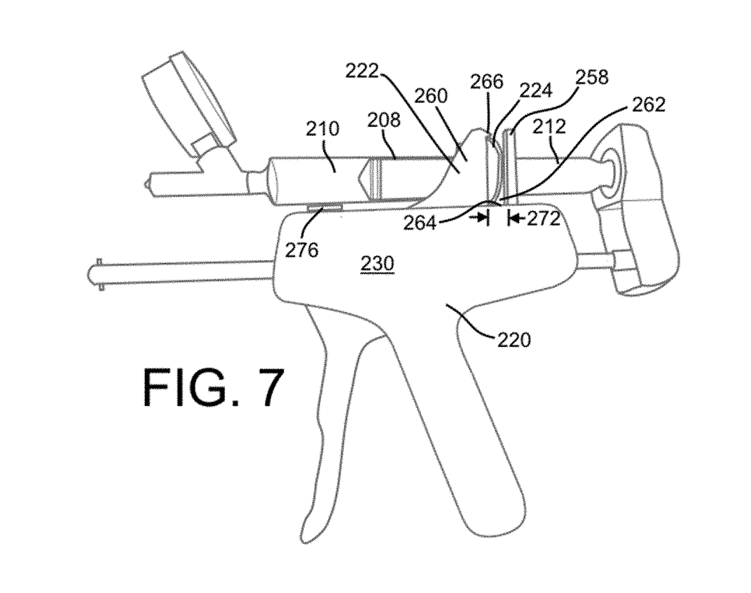

Referring to FIG. 7, a bracket 222 of an actuation device 220 may have a proximal element 258 and a distal element 260 with an opening 262 formed therebetween. A bottom 264 of the opening 262 may be formed by a top surface of a body 230 of the actuation device 220, by a connection (not shown) between the proximal element 258 and the distal element 260, and/or another suitable structure. The distal element 260 may have a detent 266. The detent 266 may extend into the opening 262 of the bracket 222 and may be configured to prevent a flange 224 from sliding or otherwise moving out of the opening 262. As shown, a bottom portion of the opening may have a thickness 272 that is larger than a thickness of the flange 224, which may be advantageous for quick and efficient insertion of the flange 224 into the opening. Once a barrel 210 of the syringe 208 becomes pressurized (e.g., due to the actuation device 220 providing a force on a plunger 212 of the syringe 208), the flange 224 may experience a force in the distal direction such that the flange 224 engages the distal element 260 of the bracket 222. When this occurs, the flange 224 may be relatively secure within the opening 262 since the detent 266 is positioned to block vertical motion of the flange 224. The detent 266 may be located approximately a distance from the bottom 264 of the opening 262 corresponding to a cross-sectional width of the flange 224 at a location (e.g., cross-sectional plane of the flange 224) where the flange 224 contacts the bracket 222. It is contemplated that a bottom portion of the detent 266 may define a curved or sloped surface, which may be advantageous for facilitating the positioning of the flange 224 in a particular vertical position once the flange 224 engages the distal element 260 of the bracket 222. While the detent 266 may be compatible with a collar, it is also contemplated that the detent 266 may advantageously allow for use of a relatively small original flange of an associated relatively small syringe (even without a collar).

A spacer 276 may be located between the barrel 210 of the syringe 208 and the actuation device 220. The spacer may be integral with the body 230 of the actuation device, or alternatively, it may be a separate component that is assembled to the body 230 and/or to the syringe 208. The spacer 276 may be advantageous where a relatively small syringe is used with an actuation device 220 initially designed for a larger syringe (i.e., where the larger syringe would be supported along its length without the spacer 276, but the smaller syringe 208 may not be). The spacer 276 may be made of a compliant material, and may be placed between the barrel 210 of the syringe 208 and the body 230 of the actuation device 220 after the syringe 208 is secured. It is contemplated that the spacer 276 may not be required, and that the syringe 208 may be sufficiently supported without the spacer 276.

The figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

We claim:

1. A collar for securement to a barrel of a syringe, the barrel having an outer diameter and a flange extending from the outer diameter, the collar comprising:
a distal opening with an inner diameter configured to surround the outer diameter of the barrel;
a collar flange located proximal the distal opening, the collar flange having a length and a thickness greater than a length and a thickness of the flange of the barrel; and
a flange-receiving cavity extending at least partially within the collar flange and configured to receive the flange of the barrel.

2. The collar of claim 1, further comprising an outer diameter surface with a larger diameter than the outer diameter of the barrel of the syringe.

3. The collar of claim 1, wherein the collar flange is a first collar flange, the collar further comprising:
a first half with the first collar flange and a second half with a second collar flange, wherein the first half and the second half are discrete elements configured to secure together around the outer diameter of the syringe.

4. The collar of claim 1, wherein the collar includes an outer diameter surface with a diameter of about 29.5 mm.

5. The collar of claim 1, wherein the collar flange has a length of about 10.4 mm.

6. The collar of claim 1, wherein the collar flange has a thickness of about 3.8 mm.

7. An inflation system for an inflation catheter, the inflation system comprising:
a syringe, the syringe having a barrel with a cylindrical chamber and a plunger, the plunger having a distal end being slidable within the cylindrical chamber of the barrel, wherein a flange of the syringe extends from an outer diameter of the barrel; and
a collar, the collar having a flange-receiving cavity for receiving the flange of the syringe and an opening with an inner diameter surrounding the outer diameter of the barrel.

8. The inflation system of claim 7, wherein the collar includes an outer diameter surface with a larger diameter than an outer diameter of the barrel of the syringe.

9. The inflation system of claim 7, wherein the collar includes a first half with a first collar flange and a second half with a second collar flange, wherein the first half and the second half are discrete elements configured to secure together.

10. The inflation system of claim 7, wherein the collar includes a collar flange, and wherein the collar flange is configured to be received by an opening of a bracket of an actuation device.

11. The inflation system of claim 10, wherein the inflation system further comprises the bracket, and wherein the bracket includes a detent extending into the opening of the bracket.

12. The inflation system of claim 10, further comprising a spacer configured to be positioned between the barrel of the syringe and a top surface of the actuation device.

13. The inflation system of claim 7, wherein the collar includes an outer diameter surface with a diameter of about 29.5 mm.

14. The inflation system of claim 7, wherein the collar includes a collar flange, and wherein the collar flange has a length of about 10.4 mm.

15. The inflation system of claim 7, wherein the collar includes a collar flange, and wherein the collar flange has a thickness of about 3.8 mm.

16. An inflation system of an inflation catheter, the inflation system comprising:
a syringe, the syringe having a barrel with a cylindrical chamber and a plunger, the plunger having a distal end being slidable within the cylindrical chamber of the barrel, wherein a flange of the syringe extends from an outer diameter of the barrel;
a collar with a collar flange, the collar having an opening configured to receive an outer diameter of the barrel of the syringe; and
an actuation device configured to move the plunger with respect to the barrel of the syringe, the actuation device including a bracket with a proximal element, a distal element, and an opening between the proximal element and the distal element,
wherein the collar flange is received by the opening of the bracket.

17. The inflation system of claim 16, wherein the distal element of the bracket includes a detent extending into the opening of the bracket.

18. The inflation system of claim 16, wherein the collar includes a flange-receiving cavity for receiving a flange of the syringe.

19. The inflation system of claim 16, further comprising a spacer located between a top surface of the actuation device and the barrel of the syringe.

20. The inflation system of claim 16, wherein the collar flange is a first collar flange, the collar comprising:
a first half with the first collar flange and a second half with the second collar flange, wherein the first half and the second half are discrete elements configured to secure together.
